(19) United States
(12) Patent Application Publication
(10) Pub. No.: US 2006/0211981 A1
(43) Pub. Date: Sep. 21, 2006

(54) MEDICAL TREATMENT PROCEDURE AND SYSTEM IN WHICH BIDIRECTIONAL FLUID FLOW IS SENSED

(75) Inventors: Douglas Ray Sparks, Whitmore, MI (US); Nader Najafi, Ann Arbor, MI (US)

Correspondence Address: HARTMAN & HARTMAN, P.C., 552 EAST 700 NORTH, VALPARAISO, IN, 46383, US

(73) Assignee: INTEGRATED SENSING SYSTEMS, INC., Ypsilanti, MI (US)

(21) Appl. No.: 11/306,395

(22) Filed: Dec. 27, 2005

Related U.S. Application Data
(60) Provisional application No. 60/639,406, filed on Dec. 27, 2004.
(60) Provisional application No. 60/721,220, filed on Sep. 29, 2005.

(51) Int. Cl. A61M 37/00; A61M 1/00; A61M 3/00

(52) U.S. Cl. 604/44; 604/29; 604/6.09

(57) ABSTRACT

A medical treatment procedure and system that makes use of a bidirectional flow sensor unit to monitor, detect, and control the flow of one or more fluids to and from a patient. The sensor unit measures both flow rate and flow direction of a fluid of a conduit through which a first fluid flows to or from the patient in a first direction, and through which it is possible that the first fluid or a second fluid may flow in a reverse direction through the conduit from or to, respectively, the patient. The sensor unit measures the flow rate of the first fluid as the first fluid flows through the bidirectional flow sensor unit, and senses if the first fluid or the second fluid flows through the bidirectional flow sensor unit in the reverse direction. A signal is relayed to indicate the occurrence of a reverse flow condition.

### CROSS REFERENCE TO RELATED APPLICATIONS

This application claims the benefit of U.S. Provisional Application No. 60/639,406, filed Dec. 27, 2004, and U.S. Provisional Application No. 60/721,220, filed Sep. 29, 2005. The contents of these prior applications are incorporated herein by reference.

### BACKGROUND OF THE INVENTION

The present invention generally relates to medical treatment systems that deliver fluids to a patient. More particularly, this invention relates to a bidirectional flow sensing device for use in medical treatment systems adapted to deliver one or more fluids to perform an infusion, transfusion, perfusion, catheterization, dialysis, respiration, or anesthetization procedure, and which may unintentionally or intentional entail bidirectional flow through a conduit delivering the fluid.

A variety of drug infusion pumps, blood perfusion systems, dialysis, and catheter systems have been developed over the years that make use of elastomeric, gravity fed, syringe, electrical, and mechanical pumps. Valves and flow sensors have been incorporated into some infusion pump designs to improve dosage accuracy and control the flow of drugs from the system. Micromachined flow sensors, valves, and pumps have been developed that can replace traditional flow sensors, valves, and pumps used in drug delivery systems. A notable example of a micromachined flow sensor is commonly-assigned U.S. Pat. No. 6,477,901 to Tadigadapa et al.

Various medical treatments entail intentionally delivering or withdrawing a fluid from a patient through a conduit, examples of which include but are not limited to drug infusion, blood transfusion, perfusion, catheterization, kidney dialysis, respiration assistance and monitoring, and delivery of anesthetics. In each case, a fluid (e.g., a drug, blood, urine, oxygen, expiration, anesthetic, etc.) is passed through a conduit to or from a patient. Such treatments may, either intentionally or unintentionally, result in both delivery and withdrawal of fluids. Examples of intentional withdrawal and delivery of fluids include dialysis, respiration assistance with oxygen, delivery of anesthetics, and retrograde infusion, transfusion, and perfusion procedures in which a body fluid is withdrawn, treated or supplemented, and then returned to the body. Retrograde drug infusion can also be employed to delivery multiple drugs that may otherwise be incompatible. Examples of unintentional withdrawal and delivery of fluids include drug infusion procedures during which, for one reason or another, body fluids are withdrawn through the conduit intended to delivery the drug, in which case bidirectional fluid flow occurs within the conduit.

A number of medical problems may arise during procedures in which fluids are both withdrawn and delivered to a patient, such as air embolisms and high blood pressure as a result of inadequate control and accuracy of fluid flow, especially in neonatal and pediatric applications. In the past, flow rate measurements have been typically performed by ultrasonic flow sensors, optical sensors, and volumetric containers. To reduce the risk that a fluid will be improperly delivered or withdrawn, additional sensors, equipment, and procedures have been used to monitor the efficiency and progress of such procedures, including pressure sensors, air bubble detectors, temperature monitors, etc., each usually as a separate individual sensor. However, accurate flow measurement remains a challenge, particularly if bidirectional flow is or may be encountered.

### BRIEF SUMMARY OF THE INVENTION

The present invention provides a medical treatment procedure and system that make use of a bidirectional flow sensor to monitor, detect, and/or control the flow of fluids to and from a patient, as in the case of certain infusion, transfusion, and perfusion procedures, dialysis, respiration assistance and/or monitoring, and delivery of anesthetics. More particularly, the invention utilizes a bidirectional flow sensor unit to measure both flow rate and flow direction of a fluid. In treatments where bidirectional flow through a conduit is not desired, such as dialysis and infusion, transfusion, perfusion procedures, the bidirectional flow sensor unit can be used to detect, measure (if desired), and provide an appropriate warning of reverse (retrograde) flow of a fluid being delivered or withdrawn. In cases where both withdrawal and delivery of one or more fluids are desired, such as retrograde infusion, transfusion and perfusion procedures, respiration, and anesthetization, the bidirectional flow sensor unit allows the flow rate and flow direction to be measured and, when coupled with appropriate fluid control devices, controlled.

The procedure of this invention includes placing a conduit for flowing a first fluid to or from a living body in a first direction and through which it is possible that the first fluid or a second fluid may flow in a reverse direction through the conduit from or to, respectively, the living body. A bidirectional flow sensor unit is fluidically coupled to the conduit so that the first fluid and optionally the second fluid are able to flow therethrough in the first and reverse directions. The bidirectional flow sensor unit comprises means for sensing the flow rate and flow direction of the first fluid and optionally the second fluid flowing through the bidirectional flow sensor unit. The sensing means is then used to measure the flow rate of the first fluid as the first fluid flows through the bidirectional flow sensor unit, and sense if the first fluid or the second fluid flows through the bidirectional flow sensor unit in the reverse direction. A signal is then relayed to indicate the occurrence of the first fluid or the second fluid flowing through the bidirectional flow sensor unit in the reverse direction.

The system of this invention includes a conduit placed for flowing a first fluid to or from a living body in a first direction and through which it is possible that the first fluid or a second fluid may flow in a reverse direction through the conduit from or to, respectively, the living body. A bidirectional flow sensor unit is fluidically coupled to the conduit so that the first fluid and optionally the second fluid are able to flow therethrough in the first and reverse directions. The bidirectional flow sensor unit comprises means for sensing the flow rate and flow direction of the first fluid and optionally the second fluid flowing through the bidirectional flow sensor unit. The system further includes means for relaying a signal indicating the occurrence of the first fluid or the second fluid flowing through the bidirectional flow sensor unit in the reverse direction.

A significant advantage of this invention is that various sensors and devices previously required in medical treatment procedures and systems to measure fluid flow rates and monitor or safeguard against retrograde flow can be replaced by a bidirectional flow sensor unit capable of accurately sensing both. In the context of a treatment where bidirectional flow through the same conduit is not desired, such as dialysis and infusion, transfusion, perfusion procedures, the bidirectional flow sensor unit can be used to detect, measure (if desired), and provide an appropriate warning of reverse (retrograde) flow of a fluid being delivered or withdrawn. In the context of a treatment where both withdrawal and delivery of one or more fluids are desired, such as retrograde infusion, transfusion and perfusion procedures, respiration, and anesthetization, the bidirectional flow sensor unit allows the flow rate and flow direction to be measured, monitored, and, if coupled with appropriate fluid control devices, controlled.

Other objects and advantages of this invention will be better appreciated from the following detailed description.

### BRIEF DESCRIPTION OF THE DRAWINGS

FIG. 1 is a schematic representation of a fluid delivery system mounted to an intravenous pole and adapted to infuse, transfuse, or perfuse a drug, blood, or other bodily or medicinal fluid through an intravenous tube in accordance with certain embodiments of the invention.

FIGS. 2 and 3 are schematic representations of systems adapted to assist and/or monitor respiration and/or deliver an anesthetic in accordance with additional embodiments of the invention.

FIG. 4 is a perspective view of a bidirectional flow sensing unit for use in the treatment system of FIG. 1.

FIGS. 5 and 6 are perspective and cross-sectional views, respectively, of a Coriolis-type mass flow rate sensor suitable for use in the sensing unit of FIG. 2.

FIGS. 7 through 9 illustrate the Coriolis effect on the sensor of FIGS. 4 and 5.

### DETAILED DESCRIPTION OF THE INVENTION

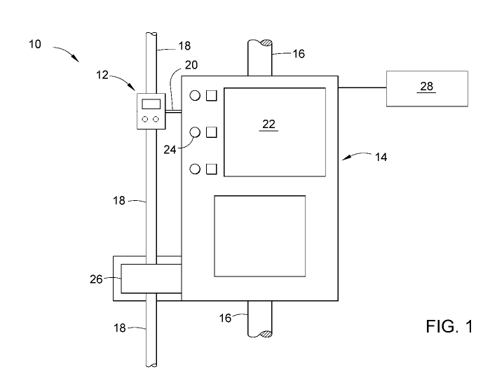

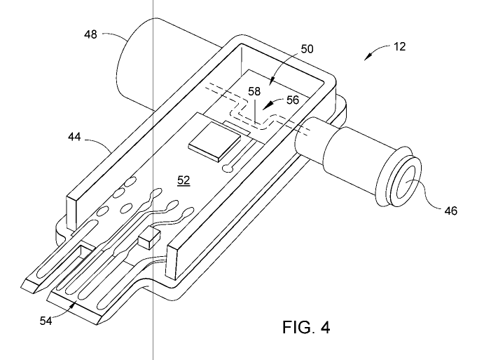

FIG. 1 represents a medical treatment system 10 that can be employed in an infusion, transfusion, perfusion, or dialysis procedure. The system 10 is shown as comprising a console 14 mounted to a pole 16, alongside which a tube 18 is secured for delivering or withdrawing a fluid from a patient. As an example, the tube 18 may be an intravenous (IV) tube or other suitable conduit suitable for the treatment being performed, and terminated with any suitable delivery device, such as a cannula, catheter, etc. As will be appreciated by those skilled in the art from the following discussion, the fluid may be a medicinal drug, nutritional solution, or body fluid if the procedure is an infusion, transfusion, perfusion treatment, blood if the procedure is a dialysis treatment, etc. A sensor unit 12 is fluidically in line with the tube 18 and communicates with the console 14 through a connector 20. According to a preferred embodiment of the invention, the sensing unit 12 is a bidirectional flow sensor unit 12, such as of a type represented in FIG. 4 and described in greater detail below. Suitable electronic circuitry (not shown) for communicating with the sensor unit 12 may be located on the unit 12 or console 14. The console 14 is equipped with a display 22 for providing a visual indication of the operation of the system 10. An AC power cord (not shown) or rechargeable battery (not shown) may be employed to power both the console 14 and the sensor unit 12. The console 14 is also shown as being equipped with audible and visual alarms 24 for warning nearby caregivers of any errors encountered during operation of the system 14, e.g., an improper flow rate, flow direction, or fluid density for the fluid flowing through the tube 18, as well as other appropriate notifications that can be initiated by the sensor unit 12. The console 14 can be further equipped with other warning indicators and controls, such as a low battery warning light, reset/confirm buttons, etc. A flow device 26 is shown as being mounted to the side of the console 14. Depending on the particular operation mode of the system 10, the flow device 26 may be a shut-off valve for stopping flow of the fluid through the tube 18 in response to the output of the sensor unit 12 or console 14, or a pump to induce and/or reverse flow through the tube 18. The console 14 is preferable connected to a computer 28 by which the operation and status of the console 14 can be controlled and monitored. While the sensor unit 12, console 14, and computer 28 are represented as physically interconnected for communication, it is also within the scope of this invention that wireless communication techniques could be used, including IR, RF, optical, magnetic, etc.

A preferred configuration for the sensing unit 12 of this invention is represented in FIG. 4. The unit 12 is shown as comprising a housing 44 adapted for inline installation, though other configurations are also possible and within the scope of this invention. The housing 44 is formed to have a fluid inlet 46 and outlet 48, both of which can be adapted for a fluidic connection through such fittings as a Luer, threaded, compression, barbed, lock or other type of fitting. The housing 44 contains a sensor 50 and electronic circuitry 52 located and enclosed within a cavity defined within the housing 44 and closable with a cover (not shown). The sensor 50 is the structure through which the fluid flowing through the tube 18 is sensed, and is therefore adapted to provide a measurable response to various properties of the fluid, which in accordance with the invention include at least the flow rate and flow direction of the fluid through the sensor unit 12. The circuitry 52 is preferably configured to communicate with and control the sensor 50 and output information regarding the operation of the sensing unit 12 to the console 14. The unit 12 further includes an electrical connector 54 by which the circuitry 52 can be coupled to the console 14, as well as to a computer or another suitable electronic device capable of controlling and receiving signals from the sensing unit 12. As noted above, an alternative to the connector 54 is a wireless communication device. Power for the sensor 50 and circuitry 52 can be provided with a battery (not shown) within the housing 44, delivered through a cable connected via the connector 54, or delivered telemetrically using known tele-powering techniques. The console 14 is equipped with a display 22 for providing a visual indication of the operation of the system 10. An AC power cord (not shown) or rechargeable battery (not shown) may be employed to power both the console 14 and the sensor unit 12. Similar to the console 14, the sensor unit 12 can be equipped with a display, audible and visual alarms in response to the operation of the unit 12, power indicators, reset/confirm buttons, etc.

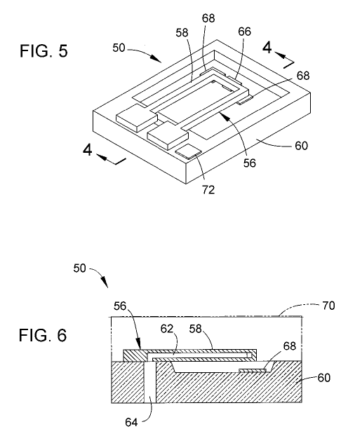

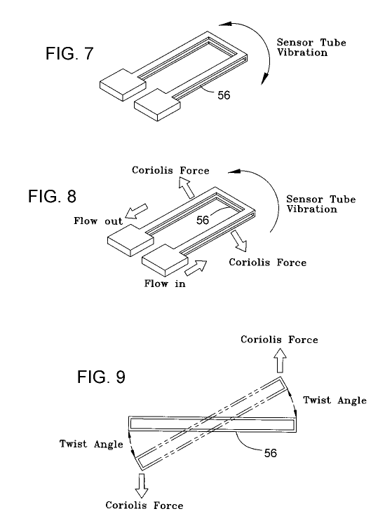

The sensor 50 is represented as comprising a tube 56 that serves as a conduit through which the fluid flows as it flows between the inlet 46 and outlet 48 of the housing 44. In a preferred embodiment of the invention, the sensor 50 and its tube 56 are part of a Coriolis mass flow sensor. FIGS. 5 and 6 depict a preferred Coriolis mass flow sensor 50 taught in commonly-assigned U.S. Pat. No. 6,477,901 to Tadigadapa et al., whose discussion of the construction and operation of a Coriolis flow sensor is incorporated herein by reference. In Tadigadapa et al., wafer bonding and silicon etching techniques are used to micromachine the tube 56 and its freestanding portion 58, which is suspended over a silicon substrate 60. While the freestanding portion 58 of the tube 56 is represented as generally U-shaped, other shapes, both simpler and more complex, are within the scope of this invention. In accordance with Tadigadapa et al, the freestanding portion 58 can be vibrated in a direction perpendicular to the underlying surface of the substrate 60. Fluid flows through an internal passage 62 within the tube 56, and enters and exits the tube 56 through fluid inlet and outlet passages (one of which is identified with reference number 64 in FIG. 6) provided in the substrate 60. During half of the vibration cycle in which the freestanding portion 58 of the tube 56 moves upward, the freestanding portion 58 has upward momentum as the fluid travels around the tube bends, and the fluid flowing out of the freestanding portion 58 resists having its vertical motion decreased by pushing up on that part of the freestanding portion 58 nearest the fluid outlet. The resulting force causes the freestanding portion 58 of the tube 56 to twist. As the tube 56 moves downward during the second half of its vibration cycle, the freestanding portion 58 twists in the opposite direction. This twisting characteristic, illustrated in FIGS. 7 through 9, is referred to as the Coriolis effect. As explained in Tadigadapa et al., the degree to which the freestanding portion 58 of the tube 56 twists (deflects) during a vibration cycle as a result of the Coriolis effect can be correlated to the mass flow rate of the fluid flowing through the tube 56. In addition, the density of the fluid is proportional to the natural frequency of the fluid-filled freestanding portion 58, such that controlling the vibration of the portion 58 to maintain a frequency at or near its resonant frequency will result in the vibration frequency changing if the density of the fluid flowing through the tube 56 changes.

The resonant frequency of the freestanding tube portion 58 is determined in part by its mechanical design (shape, size, construction and materials). Suitable frequencies are in the range of 1 kHz to over 100 kHz, depending on the particular fluid being analyzed. Under most circumstances, frequencies above 10 kHz, including ultrasonic frequencies (those in excess of 20 kHz), will be preferred. The amplitude of vibration is preferably adjusted through means used to vibrate the tube portion 58. For this purpose, FIG. 5 shows an electrode 66 located beneath the freestanding portion 58 on the surface of the substrate 60. In the embodiment shown, the tube 56 serves as an electrode (e.g., is formed of doped silicon) that is capacitively coupled to the electrode 66, enabling the electrode 66 to electrostatically drive the freestanding portion 58. However, it is foreseeable that the tube 56 could be formed of a nonconductive material, requiring a separate electrode formed on the freestanding portion 58 opposite the electrode 66 for vibrating the freestanding portion 58 electrostatically. Furthermore, the freestanding portion 58 could be driven capacitively, piezoelectrically, piezoresistively, acoustically, ultrasonically, magnetically, optically, or by another actuation technique. Also shown in FIGS. 5 and 6 are sensing electrodes 68 for providing feedback to enable the vibration frequency and amplitude to be controlled with the circuitry 52 within the sensing unit 12. While capacitive sensing is preferred, the sensing elements 68 could sense the proximity and motion of the freestanding portion 58 in any other suitable manner.

In order to provide a temperature-sensing capability, the sensor 50 is shown in FIG. 5 as including an on-chip thin film temperature sensor 72, such as a resistance temperature detector (RTD), in close proximity to the resonating tube 56. The temperature sensor 72 is shown integrated onto the same substrate 60 as the tube 56 to provide an accurate fluid temperature output, which in addition to providing useful temperature data also enables temperature to be factored into the fluid density measured by the sensor 50. Alternatively, a temperature sensing capability can be achieved by fabricating a second cantilevered tube on the substrate 60. According to commonly-assigned U.S. Pat. No. 6,647,778 to Sparks, vibrating the cantilevered tube at resonance enables the tube to measure the temperature of the fluid flowing therethrough on the basis that the Young's and shear modulus of the materials used to form the tube change with temperature, causing the resonant frequency of the tube to detectably shift with temperature.

FIG. 6 schematically represents the micromachined tube 56 enclosed by a cap 70 bonded or otherwise attached to the substrate 60. In a preferred embodiment, the bond between the cap 70 and substrate 60 is hermetic, and the resulting enclosure is evacuated to enable the freestanding portion 58 to be driven efficiently at high Q values without damping. A suitable material for the cap 70 is silicon, allowing silicon-to-silicon bonding techniques to be used, though other cap materials and bonding techniques are possible and within the scope of the invention.

As discussed above and represented in FIGS. 7 through 9, the direction of twist of the freestanding portion 58 depends on the direction of fluid flow through the tube 56. In accordance with this invention, the circuitry 52 and sensing elements 68 of the sensor 50 cooperate to sense the direction of twist of the tube 56 relative to the drive electrode 66 or phase differences between the laterally opposite side portions of the tube 56 or other parts of the tube resulting from the Coriolis effect, which can then be correlated to the direction of flow through the tube 56 and, therefore, the sensor unit 12 containing the tube 56. As such, it can be appreciated that the resonating tube flow sensor 50 is well suited for use in the sensing unit 12 of this invention for the purpose of sensing both flow rate and flow direction, though it is foreseeable that other types of flow sensors could be employed, such as hot-wire, thin-film, drag force, ultrasonic, pressure, or another type of flow sensor. However, particularly advantageous aspects of the resonating tube sensor 50 include its very small size, its ability to precisely measure extremely small amounts of fluids, and, of particular interest to the present invention, its ability to sense bidirectional fluid flow, in contrast to prior art flow sensors that, if not inherently bidirectional, would require the use of more than one flow sensor per tube 18 to provide a bidirectional capability. Furthermore, the preferred flow sensor 50 can attain flow rate measurement accuracies of under ±1%, in contrast to other types of infusion pumps whose accuracies can range from about ±15% for volumetric pumps and ±3% for syringe pumps. While the high cost and the high flow rate requirements for prior art Coriolis-type flow sensors have restricted their use in the drug delivery arena, the flow sensor 50 is able to sense the extremely low flow rates (e.g., less than 1 ml/hr) required by infusion therapy applications, and can be used to sense the flow rates associated with the treatment system 10 of FIG. 1.

From the above, it can be appreciated that sensor units 12 equipped with the sensor 50 can be advantageously employed in the treatment system 10 of FIG. 1. If a fluid is being delivered, the sensing unit 12 is placed downstream of any type of drug delivery device, including but not limited to an IV bag, IV set, peristaltic pump, syringe, syringe pump, electromechanical pump, pressurized pump, implanted pump, etc., enabling the flow rate of the fluid to be accurately monitored to ensure a proper amount of fluid is delivered. Dose and dose rates can also be calculated based on the flow rate measured with the sensor unit 12. With the addition of one or more sensor units 12, multiple fluids can be delivered with the treatment system 10. In addition to sensing the flow rate of the fluid flowing through the tube 18, and therefore being administered to or withdrawn from a patient, the sensor 50 is able to sense in which direction the fluid is flowing, either in the intended direction or the reverse direction, by sensing the direction of twist of the freestanding portion 58 of the sensor tube 56. As such, if bidirectional flow through the fluid tube 18 is not desired, such as during dialysis and infusion, transfusion, perfusion procedures, the sensor unit 12 can be used to detect, measure (if desired), and provide an appropriate warning of reverse (retrograde) flow of the fluid occurs, such as with the alarm 24 of the console 14. Alternatively, if the tube 18 is intended to selectively withdraw and deliver of one or more fluids, such as during a retrograde infusion, transfusion or perfusion procedure, respiration assistance and monitoring, and delivery of anesthetics, the sensor unit 12 allows the flow rate and flow direction of the one or more fluids to be measured, monitored, and, if coupled with appropriate fluid control devices, controlled. In view of these benefits, the sensor unit 12 of this invention can be employed to improve the safety of a variety of medical treatment procedures, especially for neonatal and pediatric applications in which dose sensitivity is particularly critical. The sensor unit 12 also enables multiple drugs to be delivered with a single conduit 18 through the ability to detect barrier solutions delivered between incompatible drugs based on changes in density (as indicated by changes in the resonant frequency of the sensor tube 56).

The above-noted density and temperature-sensing capabilities of the sensing unit 12 can also be utilized with the present invention to sense and monitor the specific gravity/density of the fluid to confirm that the correct fluid, drug concentration, etc., is being delivered or withdrawn, as well as detect the presence of undesired components in the fluid. In particular, the sensing unit 12 can be sufficiently sensitive to detect occlusions and fine air bubbles that could cause air embolisms, as reported in commonly-assigned U.S. patent application Ser. Nos. 10/248,839 and 10/708,509.

Because micromachining technologies are employed to fabricate the sensor tube 56, the size of the tube 56 can be extremely small, such as lengths of about 0.5 mm and cross-sectional areas of about 250 square micrometers, with smaller and larger tubes also being within the scope of this invention. Because of the ability to produce the sensor tube 56 at such miniaturized sizes, the sensor unit 12 can be used to process very small quantities of fluid for analysis. However, because miniaturization can render the sensor 50 unsuited for applications in which measurements of properties are desired for a fluid flowing at relatively high flow rates, the sensor 50 can be configured to have an internal bypass passage in accordance with the teachings of commonly-assigned U.S. patent application Ser. No. 11/164,374, whose teachings regarding the fabrication of bypass passages are incorporated herein by reference.

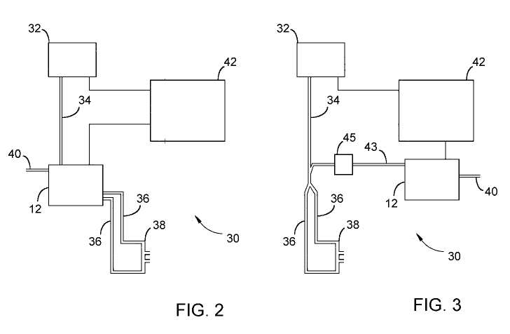

Illustrated in FIG. 2 is another system 30 configured in accordance with this invention to employ the bidirectional flow sensor unit 12. The system 30 differs from the system 10 of FIG. 1 in the manner in which it is specifically adapted to both deliver and withdraw one or more fluids within the same conduit. As represented, the system 30 is adapted to assist and/or monitor the respiration of a patient, including such procedures as supplying and monitoring supplemental respiratory oxygen, monitoring and/or preventing sleep apnea, and delivering an anesthetic to a patient. A source 32 of a breathable gas mixture, oxygen, or anesthetic is shown fluidically interconnected with the bidirectional flow sensor unit 12 of this invention through a suitable conduit 34. In turn, the sensor unit 12 is connected through a pair of tubes 36 to a cannula 38, which is represented as being a nasal cannula though other delivery devices could be used, such as a throat, mouth, or trachea cannula. Flow of the gas mixture, oxygen, or anesthetic from the source 32 is preferably regulated with a suitable device (not shown) controlled with a controller 42 that also communicates with the sensor unit 12. As a patient inhales through the cannula 38, the gas mixture, oxygen, or anesthetic is drawn through the sensor unit 12 and tubes 36. During exhalation, the patient's expiration may also be exhaled through the tubes 36 and the sensor unit 12 before being exhausted through an outlet 40 on the sensor unit 12, with the result that the sensor unit 12 is not only able to sense flow rate, but also detect the change in flow direction and, if so desired, provide an appropriate output, such as a visual or audible signal generated on the sensor unit 12 or by the controller 42, indicating a change in flow direction and therefore the completion of a respiration cycle. Because of the typically limited flow capacity of its sensor tube 56, the sensor unit 12 used in the embodiment of FIG. 2 is preferably configured with a bypass passage such that only a fraction of the gases being inhaled and exhaled passes through the sensor tube 56.

FIG. 3 shows another embodiment of the system 30, in which the sensor unit 12 is coupled to the conduit 34 and tubes 36 with a bypass tube 43 connected with a splitter on the conduit 34. The bypass tube 43 is equipped with a filter 45 that prevents bacteria, viruses, etc., exhaled by the patent from contaminating the sensor unit 12. In this manner, the conduit 34, tubes 36, cannula 38, bypass tube 43, and filter 45 constitute a disposable unit while the sensor unit 12 is reusable.

With each embodiment of FIGS. 2 and 3, the flow rates of the inhalation and exhalation of the patient can be monitored, as well as the frequency of the patient's breaths as sensed by a change in the direction of flow through the sensor unit 12. The temperature sensor 72 on the sensor 50 further permits the temperature of the patient's exhalation to be monitored. Because of the ability of the sensor unit 12 to measure density, the sensor unit 12 is also capable of monitoring the gas mixtures inhaled and exhaled by the patient.

In view of the foregoing, it can be appreciated that the present invention is also applicable to other treatment systems in which one or more fluids are delivered to or withdrawn from the human body, including retrograde (reverse) infusion, transfusion, and perfusion procedures. In such applications, both the delivery and withdrawal of the fluids can be controlled in a closed-loop system through the fluid sensor 12, controller 42, and appropriate devices under the control of the controller 42, such as valves, pumps, motors, fluid actuators, etc.

While the invention has been described in terms of a preferred embodiment, it is apparent that other forms could be adopted by one skilled in the art. Therefore, the scope of the invention is to be limited only by the following claims.

What is claimed is:

1. A medical treatment procedure comprising:
placing a conduit for flowing a first fluid to or from a living body in a first direction and through which it is possible that the first fluid or a second fluid may flow in a reverse direction through the conduit from or to, respectively, the living body;
fluidically coupling a bidirectional flow sensor unit to the conduit so that the first fluid and optionally the second fluid are able to flow therethrough in the first and reverse directions, the bidirectional flow sensor unit comprising means for sensing the flow rate and flow direction of the first fluid and optionally the second fluid flowing through the bidirectional flow sensor unit;
measuring with the sensing means the flow rate of the first fluid as the first fluid flows through the bidirectional flow sensor unit in the first direction, and sensing with the sensing means if the first fluid or the second fluid flows through the bidirectional flow sensor unit in the reverse direction; and
relaying a signal indicating the occurrence of the first fluid or the second fluid flowing through the bidirectional flow sensor unit in the reverse direction.

2. The medical treatment procedure according to claim 1, wherein the medical treatment procedure is a treatment chosen from the group consisting of drug infusion, transfusion, perfusion, catheterization, dialysis, respiration assistance, respiration monitoring, and anesthetization.

3. The medical treatment procedure according to claim 1, wherein the first fluid is chosen from the group consisting of drugs, blood, nutrients, urine, oxygen, expiration gases of the living body, and anesthetic gases.

4. The medical treatment procedure according to claim 1, wherein the medical treatment procedure is a drug infusion treatment, the first fluid is a drug, and the second fluid is a bodily fluid from the living body.

5. The medical treatment procedure according to claim 1, wherein the medical treatment procedure is a blood transfusion treatment and the first and second fluids are blood.

6. The medical treatment procedure according to claim 1, wherein the medical treatment procedure is a perfusion treatment, the first fluid is a drug, and the second fluid is a bodily fluid from the living body.

7. The medical treatment procedure according to claim 1, wherein the medical treatment procedure is a dialysis treatment and the first and second fluids are blood.

8. The medical treatment procedure according to claim 1, wherein the medical treatment procedure involves at least one of monitoring and assisting the respiration of the living body, the first fluid is oxygen, and the second fluid is expiration gases of the living body.

9. The medical treatment procedure according to claim 1, wherein the medical treatment procedure is anesthetization, the first fluid is an anesthetic, and the second fluid is expiration gases of the living body.

10. The medical treatment procedure according to claim 1, further comprising communicating the flow rate and flow direction sensed by the sensing means to a remote unit.

11. The medical treatment procedure according to claim 1, wherein the sensing means comprises:
a tube comprising a freestanding tube portion through which the fluid flows;
means for vibrating the freestanding tube portion of the tube at a resonant frequency thereof that varies with the density of the fluid flowing therethrough, the Coriolis effect causing the freestanding tube portion to twist in either a first or second twist direction while being vibrated at resonance, the freestanding tube portion exhibiting a degree of twist that varies with the mass flow rate of the fluid flowing therethrough, the freestanding tube portion twisting in the first twist direction when the first fluid flows through the bidirectional flow sensor unit in the first direction, the freestanding tube portion twisting in the second twist direction if the first fluid or the second fluid flows through the bidirectional flow sensor unit in the reverse direction; and
means for sensing movement of the freestanding tube portion of the tube, the movement-sensing means producing a first output signal based on the degree of twist of the freestanding tube portion and a second output signal indicative of the direction of twist of the freestanding tube portion.

12. The medical treatment procedure according to claim 1, wherein the sensing means is sufficiently sensitive to the density of the first fluid to detect air bubbles in the first fluid flowing through the bidirectional flow sensor unit.

13. A medical treatment system for performing a medical treatment procedure, the medial treatment system comprising:
a conduit placed for flowing a first fluid to or from a living body in a first direction and through which it is possible that the first fluid or a second fluid may flow in a reverse direction through the conduit from or to, respectively, the living body;
a bidirectional flow sensor unit fluidically coupled to the conduit so that the first fluid and optionally the second fluid are able to flow therethrough in the first and reverse directions, the bidirectional flow sensor unit comprising means for sensing the flow rate and flow direction of the first fluid and optionally the second fluid flowing through the bidirectional flow sensor unit; and
means for relaying a signal indicating the occurrence of the first fluid or the second fluid flowing through the bidirectional flow sensor unit in the reverse direction.

14. The medical treatment system according to claim 13, wherein the medical treatment procedure is a treatment chosen from the group consisting of drug infusion, transfusion, perfusion, catheterization, dialysis, respiration assistance, respiration monitoring, and anesthetization, and the first fluid is chosen from the group consisting of drugs, blood, nutrients, urine, oxygen, expiration gases of the living body, and anesthetic gases.

15. The medical treatment system according to claim 13, wherein the medical treatment procedure involves at least one of monitoring and assisting the respiration of the living body, the first fluid is oxygen, and the second fluid is expiration gases of the living body.

16. The medical treatment system according to claim 15, wherein the medical treatment system further comprises a cannula affixed to one end of the conduit and means for filtering the first fluid and optionally the second fluid before entering the bidirectional flow sensor unit from the conduit, wherein the cannula, the conduit, and the filtering means constitute a disposable unit and the bidirectional flow sensor unit constitutes a reusable unit.

17. The medical treatment system according to claim 13, wherein the medical treatment procedure is anesthetization, the first fluid is an anesthetic, and the second fluid is expiration gases of the living body.

18. The medical treatment system according to claim 17, wherein the medical treatment system further comprises a cannula affixed to one end of the conduit and means for filtering the first fluid and optionally the second fluid before entering the bidirectional flow sensor unit from the conduit, wherein the cannula, the conduit, and the filtering means constitute a disposable unit and the bidirectional flow sensor unit constitutes a reusable unit.

19. The medical treatment system according to claim 13, further comprising means for communicating the flow rate sensed by the sensing means to a remote unit.

20. The medical treatment system according to claim 13, wherein the sensing means comprises:
a tube comprising a freestanding tube portion through which the fluid flows;
means for vibrating the freestanding tube portion of the tube at a resonant frequency thereof that varies with the density of the fluid flowing therethrough, the Coriolis effect causing the freestanding tube portion to twist in either a first or second twist direction while being vibrated at resonance, the freestanding tube portion exhibiting a degree of twist that varies with the mass flow rate of the fluid flowing therethrough, the freestanding tube portion twisting in the first twist direction when the first fluid flows through the bidirectional flow sensor unit in the first direction, the freestanding tube portion twisting in the second twist direction if the first fluid or the second fluid flows through the bidirectional flow sensor unit in the second direction; and
means for sensing movement of the freestanding tube portion of the tube, the movement-sensing means producing a first output signal based on the degree of twist of the freestanding tube portion and a second output signal indicative of the direction of twist of the freestanding tube portion.
